(19) United States
(12) Patent Application Publication
(10) Pub. No.: US 2011/0223654 A1
(43) Pub. Date: Sep. 15, 2011

(54) NANO-MICROFLUIDIC APPARATUS FOR CONTINUOUS REAL-TIME ANALYSIS OF TARGETS IN THIN LIQUID FILMS

(76) Inventors: Hoi-Ying N. Holman, Oakland, CA (US); Robin Miles, Danville, CA (US)

(21) Appl. No.: 12/672,251

(22) PCT Filed: Aug. 5, 2008
(86) PCT No.: PCT/US08/72253

§ 371 Date: Feb. 4, 2010

Related U.S. Application Data
(60) Provisional application No. 60/954,311, filed on Aug. 6, 2007.

(51) Int. Cl. C12M 1/42

(52) U.S. Cl. 435/288.7

(57) ABSTRACT

Nano-microfluidic devices and uses thereof are described. In particular, systems and methods are described for continuous real-time monitoring and analysis of targets in thin liquid films; such targets can include living cells and tissues. In some embodiments, nano-microfluidic devices can be utilized to observe living cells in layers of thin liquid media by IR-spectroscopy.

### CROSS-REFERENCE TO RELATED APPLICATIONS

This application claims priority to U.S. Provisional Application 60/954,311 filed Aug. 6, 2007, which is incorporated herein by reference in its entirety for all purposes.

### STATEMENT REGARDING FEDERALLY SPONSORED R&D

This invention was made with government support under Contract DE-AC02-05CH11231 and W-7405-ENG-48 awarded by the U.S. Department of Energy, and under Contract No. DE-AC52-07NA27344 awarded by the U.S. Department of Energy and the NNSA. The government has certain rights in this invention.

### FIELD OF THE INVENTION

The present invention relates to nano-microfluidic devices and uses thereof. In particular, systems and methods are described for continuous real-time analysis of targets in thin liquid films; such targets can include living cells and tissues.

### BACKGROUND

Many different techniques are currently used to analyze biological molecules. For example, fluorescent or radioactive tags can be linked to biomolecules and thereafter tracked as they traverse a biological pathway. Unfortunately, it can be challenging to track real-time changes in biomolecules within a living organism. For example, the growth media required to support the organism may reduce the signal propagated from the label. In simpler systems, the presence of even the smallest amount of liquid may prevent a label from being detected. Accordingly, several techniques have been developed to help monitor and analyze biomolecules.

Infrared (IR) spectroscopy is a technique that enables the detailed molecular, chemical and structural analysis of compounds. It can be used to identify compounds and investigate sample composition. The IR portion of the electromagnetic spectrum can be divided into three ranges: the far-IR (1000-30 μm), mid-IR (30-1.4 μm), and near-IR (1.4-0.8 μm). IR spectroscopy exploits the fact that molecules have specific IR-active vibrational modes, namely, specific frequencies at which they rotate or vibrate. These IR-active vibrational modes correspond to discrete energy levels which can be measured using IR light and thus reveal the molecular structure of a sample.

Many biomolecules, such as nucleic acids, proteins, and lipids, have characteristic and well-defined IR-active vibrational modes (Parker, F. S.: Applications of IR Spectroscopy in Biochemistry, Biology, and Medicine, New York, Plenum Press, 1971; Mantsch, H. H. and Chapman, D.: IR Spectroscopy of Biomolecules, New York, Wiley-Liss, 1996; Stuart, B. and Ando, D. J.: Biological Applications of IR Spectroscopy, Chichester, N.Y., Published on behalf of ACOL (University of Greenwich) by John Wiley, 1997)). With appropriate interpretation of measured IR spectra, many molecular species within a biological sample can be detected, identified, characterized, and quantified.

However, making IR measurements on living cells and tissues is highly problematic. The aqueous environment required to sustain living cells and tissues strongly absorbs IR light and severely limits any data that might be obtained. Moreover, existing technology does not allow the use of IR spectroscopy for long-term (hours and longer) imaging of living cells and tissues. Approaches to overcome these limitations have met with small success.

In one approach to making IR measurements on biological samples, the aqueous component of the IR spectra is subtracted from the total IR spectra of a suspended biological sample. One limitation to this technique includes the necessity that the liquid be pure, and remains constant or near-constant with time and locations. Thus this technique is not appropriate for biological samples in complex aqueous environments, such as growth media and other buffered solutions.

Another approach utilizes isotope-exchange. The incorporation of isotopes, such as deuterium, can make compounds more transparent to IR light. In one example, bacteria were grown on deuterated substrates in order to obtain measurements with a sufficient signal above background noise (Cameron, D. G., et al. (1983). Membrane isolation alters the gel to liquid-crystal transition of Acholeplasma-laidlawii-B. Science 219, 180 182). However, deuterated substrates are known to alter cellular activities and can induce cell death (Newo, A. N. S. et al. (2004). Deuterium oxide as a stress factor for the methylotrophic bacterium Methylophilus sp. 8-7741, Microbiology 73, 139 142; Pshenichnikova, A. et al. (2004). Effect of deuteration on the activity of methanol dehydrogenase from Methylophilus sp B-7741. Appl. Biochem. Microbiol. 40, 18-21).

Yet another approach to making IR measurements on biological samples utilizes attenuated-total-reflectance (ATR)-based techniques in conjunction with IR spectroscopy. ATR uses a property of total internal reflection known as the evanescent wave phenomenon. Limitations of ATR-based techniques include the need to utilize a large numbers of cells or biological particles, and measurements are limited to average surface properties with low resolution (˜1 mm).

Yet another approach utilizes attenuated-total-reflectance (ATR)-based techniques in conjunction with IR microspectroscopy. Limitations of this approach include the need to utilize an internal reflection element (IRE) tip in intimate contact with the smooth surface of a dry thin-section biological material. Moreover, measurements can have a moderate resolution only.

### SUMMARY

Some embodiments described herein relate to nano-microfluidic devices and uses thereof. In some embodiments, a nano-microfluidic system can include a platform having a substrate containing at least one channel; an aqueous layer in fluid contact with the substrate; at least one inlet in fluid communication with the aqueous layer; and at least one outlet in fluid communication with the aqueous layer. In such embodiments, the aqueous layer can contain a fluid that flows from the at least one inlet to said at least one outlet.

In certain embodiments, the at least one channel has a depth that is less than about 100 μm, less than about 50 μm, less than about 10 μm, less than about 5 μm, or less than about 1 μm. In more embodiments, the aqueous layer has a depth less than about 10 μm, less than about 5 μm, less than about 1500 nm, less than about 1000 nm, or less than about 400 nm.

In further embodiments, the inlet is fluidly coupled to an inlet reservoir. In yet further embodiments, the outlet can be fluidly coupled to an outlet reservoir.

In particular embodiments, the substrate can comprise an infrared (IR) transparent material. In more embodiments, the IR transparent material can be selected from the group consisting of diamond, ZnSe, and Si₃N4.

In some embodiments the substrate can have a coating. In some such embodiments, the coating is reflective to IR electromagnetic radiation. In more such embodiments, the coating is patterned on said substrate. In certain embodiments, the coating comprises a hydrophilic material. In such embodiments, the said coating comprises a material selected from the group consisting of titanium oxide, gold, and platinum. In other embodiments, the coating comprises a hydrophobic coating. In such embodiments, the coating comprises a material selected from the group consisting of silicone, SU-8 epoxy, and Teflon®.

In some embodiments, a stream of gas is provided above the nano-microfluidic system such that it flows above an aqueous layer. In such embodiments, the gas can be selected from the group consisting of nitrogen, argon, carbon dioxide, air, and mixtures thereof.

In particular embodiments, the substrate or nano-microfluidic system is in thermal contact with a heating/cooling source.

In certain embodiments, a nano-microfluidic system can include a source of IR electromagnetic radiation irradiating the substrate. In further embodiments, a nano-microfluidic system can also include a detector of reflected light or transmitted electromagnetic radiation.

In more embodiments, a nano-microfluidic system can include a window above the substrate. In more such embodiments, the nano-microfluidic system can also include a spacer in contact with the window and the platform. In some such embodiments, the spacer has a thickness less than 20 μm. In even more embodiments, the spacer has an adjustable thickness.

Some embodiments include a thin-liquid-film apparatus for continuous IR spectroscopy and fluorescence imaging of living cells and tissues. In one embodiment, the apparatus includes an IR spectral microscope stage incubator, comprising a multi-channel nano-microfluidic device designed to produce thin-films of moving liquid media that flow with a thickness of less than 10 μm over the surface of living cells and tissues. The thin-liquid-film can maintain mass exchange and biological activities without masking IR signals from cellular molecules. Thus, IR spectroscopy/spectromicroscopy and fluorescence/visible microscopy imaging can be combined for extended studies and measurements of biological and chemical processes within living cells and tissues.

In more embodiments, a thin-liquid-film apparatus for IR spectroscopy/spectromicroscopy and fluorescence/visible microscopy of living cells and tissues includes a controllable microscope-stage incubator with a virtual window. In such embodiments, cells and tissues can be sustained in liquid media with a depth of hundreds of nanometers to several microns. By providing a thin layer of liquid media, the apparatus can minimize the absorption of IR light while sustaining the living cells and tissues. In some embodiments, living cells and tissues can be sustained for more than 24 hours under continuous IR monitoring of biological and chemical processes.

One embodiment provides a solution to the problem of signal masking by water absorption of IR light, while maintaining the functions and/or growth of cells or biological particles. The solution is to build a cell-sustaining apparatus with a very thin layer of moving liquid which is maintained around and above the living cells or biological particles. The apparatus can incorporate either a closed system or an open flow system with a virtual window. In each case within this embodiment, the cells are sustained in a thin-film of liquid.

In one aspect, the embodiments described herein allow the non-interrupted measurement and imaging of chemical processes within living cells and tissues that are maintained in a near-native state sustainable aqueous environment.

In a further aspect, the apparatus is configured to sustain and modify cells through nano- or micro-needle injection of nano-particles or bio-particles into the cells. In another embodiment, the apparatus is configured to allow extraction of components from cells.

Another embodiment is a nano-microfluidic system that acts similar to a flow-cytometer over a broad spectrum of wavelengths, including those within the IR region. The cells or biological particles or other analytes flow down the channel of the device through the imaging area such that a large number of analytes can be analyzed in a rapid, sequential manner. Using an IR spectral signature, chemical and physiological properties of cells or other analytes can be identified and categorized. This embodiment is useful for clinical analysis of blood, urine, mucous and other clinical and environmental specimens. Such analysis is useful to identify and isolate pathogens, fetal cells, cancerous cells or any other particle of interest to a health organization.

### BRIEF DESCRIPTION OF THE DRAWINGS

FIGS. 1A and 1B are schematic diagrams showing cross-sectional and plan views, respectively, of a nano-microfluidic device.

FIGS. 2A and 2B are schematic diagrams showing cross-sectional and plan views, respectively, of a nano-microfluidic device including a window with fluid flow over the target.

FIGS. 3A and 3B are schematic diagrams showing cross-sectional and plan views, respectively, of a nano-microfluidic including a window with diffusive flow over the target.

FIG. 4 is a schematic diagram showing a cross-sectional view of a nano-microfluidic device including a window and actuator for raising the substrate having a target to modulate the depth of the aqueous layer above a target.

FIG. 5 is a schematic diagram showing a plan view of a nano-microfluidic device having wells and a reservoir in fluid contact a well.

FIG. 6 is a photograph of a nano-microfluidic device with cells in a channel. E coli reporter strains in the channel show no detectable detrimental effects for incubation in the channel. Microcolonies formed within 24 hours, and cells maintain their membrane integrity for more than 96 hours in the apparatus.

### DETAILED DESCRIPTION

Some embodiments described herein relate to nano-microfluidic devices and uses thereof. The nano-microfluidic devices described herein can be used in conjunction with a variety of techniques where analysis of biological components within a liquid phase is desirable. In one embodiment, the analysis is within a very thin liquid film phase. Many types of analysis are contemplated, including fluorescent imaging, IR spectroscopy, or any other type of imaging technique.

Generally, to observe living cells and tissues continuously in real-time for extended periods of time, e.g. several hours or more, requires that the cells or tissues are bathed in an aqueous environment. This requirement has severely limited the use many analysis techniques because water strongly absorbs the signals propagated or reflected by the cells or tissues. For example, infrared signals reflected or transmitted from target cells or tissues suspended in water are strongly absorbed at depths greater than several micrometers.

One approach would be to reduce the depth of the aqueous layer covering a target, for example, to depths about 5-10 μm or less. However, thin liquid films on flat surfaces with depths less than about 40 μm are extremely difficult to achieve due to the strong surface tension of water. Indeed, water has a high surface tension (˜72 dynes/cm) compared to the surface energy of most materials. And while the aqueous media typically used to sustain living cells and tissues may have a lower surface energy than pure water, the surface tension of such media still inhibits the formation of thin liquid films.

An additional problem is that living cells and tissues require nutrients to be carried to them, and waste products to be carried away. In small volumes, nutrients can be quickly consumed and waste products can rapidly accumulate, leading to cell or tissue death. Thus, observations of living cells and tissues in small volumes can be limited to short periods of time.

Embodiments described herein provide nano-microfluidic devices with thin liquid films. In such embodiments, living cells or tissues can be covered by or suspended within, an aqueous layer with a depth deep enough to sustain the living cell or tissue, but shallow enough to analyze the cell or tissue with minimal or no signal loss. In addition, such embodiments can provide thin liquid films that carry materials to living cells or tissues and remove products, thereby maintaining the cell or tissue for extended periods of time in which to make extended observations using IR spectroscopy.

In some embodiments, a nano-microfluidic device can include a platform that contains a substrate with at least one channel, an aqueous layer in fluid contact with the substrate and the at least one channel, and an inlet and outlet in fluid communication with the aqueous layer. In such embodiments, the aqueous layer flows from the inlet to the outlet. The substrate can comprise an IR-transparent material or an IR-opaque material. The at least one channel can have a depth of about 200 nm, 400 nm, 600 nm, 800 nm, 1 μm, about 5 μm, about 10 μm, about 20 μm, about 30 μm, about 40 μm, 50 μm, and about 100 μm. In even more embodiments, the aqueous layer can have a depth of about 100 nm, about 200 nm, about 300 nm, about 400 nm, about 500 nm, about 750 nm, about 1000 nm, about 1500 nm, about 5 μm, about 10 μm, and about 20 μm.

Particular embodiments include nano-microfluidic devices where the substrate has a coating. In such embodiments, the coating is reflective or transparent to the desired label. In one embodiment, the coating is reflective or transparent to IR electromagnetic radiation. In another embodiment the coating is reflective or transparent for particular wavelengths of light. The coating can comprise a hydrophilic material, for example, titanium oxide, gold, or platinum. The coating can comprise a hydrophobic material, such as silicone, SU-8 epoxy, and Teflon®. In certain embodiments, the coating can be patterned on the surface of the substrate.

In some embodiments, the nano-microfluidic device includes an inlet reservoir fluidly coupled to the at least one inlet. In more embodiments, the nano-microfluidic device can include an outlet reservoir fluidly coupled to the at least one outlet.

Certain embodiments include a nano-microfluidic device and a stream of gas flowing above the aqueous layer. In such embodiments, the gas can be, for example, nitrogen, argon, carbon dioxide, and air. More embodiments encompass a nano-microfluidic device including a window above the platform. The window can be IR-transparent or IR-opaque. Such embodiments can also include a spacer in contact with the window and the platform. The spacer can have a thickness less than 5 μm, less than 10 μm, less than 15 μm, less than 20 μm, and less than 40 μm; and/or the spacer can have an adjustable thickness.

In some embodiments, a nano-microfluidic device can also include a heating/cooling source in thermal contact with the platform; and/or sensors for variables such as pH, temperature, or flow rate.

In more embodiments, the system including the nano-microfluidic device has a source of IR electromagnetic radiation irradiating the platform; and/or a detector of reflected light or transmitted electromagnetic radiation.

Some embodiments include a nano-microfluidic device for the continuous IR spectroscopy and fluorescence imaging of living cells and tissues. In one embodiment, the device can include an IR spectral microscope stage incubator, comprising a multi-channel nano-microfluidic device designed to produce thin-films of moving liquid media that flow with a thickness of less than 10 μm over the surface of living cells and tissues. The thin-liquid-film maintains mass exchange and biological activities without masking IR signals from cellular molecules. This allows one to combine IR spectroscopy/spectromicroscopy and fluorescence/visible microscopy imaging for the extended studies and measurements of biological and chemical processes within living cells and tissues, using photons that span the visible through IR regions of the electromagnetic spectrum.

In another embodiment, the apparatus comprises a cell-sustaining platform having a channel or multiple channels, a spacer, and a thin aqueous layer flowing over the surface of the platform, with a virtual or actual viewing window on or over the platform, a liquid delivery means and a liquid extraction means. In another embodiment, the apparatus further comprises a temperature control means, data collection and control means, fluid delivery and extraction means, and air flow means. The apparatus allows cells to live in thin films of liquid media, therefore minimizing interfering water absorptions while keeping cells alive for more than several hours during continuous IR monitoring of biological and chemical processes. In one embodiment, the apparatus is used on a microscope stage in reflectance or transmission modes of microscopy as a fully controlled microscope-stage incubator.

In another embodiment, the apparatus can be used as a flow-cytometer having functionality over a broad spectrum of wavelengths including IR. The cells or biological particles or other analytes flow down the channel through an imaging area such that a large number of analytes can be analyzed in a rapid, sequential manner. Using an IR spectral signature, cells can be identified and categorized. Such a device can be useful for clinical analysis of blood, urine, mucous and other clinical and environmental specimens to identify and isolate pathogens, fetal cells, cancerous cells or any other particle of interest.

In a general embodiment, the apparatus can be comprised of a platform comprising a coated substrate having open channels for media flow, a spacer, and a thin aqueous layer flowing over the surface of the platform. The distance between the cell growth plane and the viewing window can be set by a spacer for the purpose of taking IR data.

The following description is provided to illustrate exemplary embodiments of the subject matter disclosed herein. Those of skill in the art will recognize that there are numerous variations and modifications of the subject matter provided herein that are encompassed by its scope. Accordingly the description of certain exemplary embodiments should not be deemed to limit the scope of the present invention.

### Platforms

In some embodiments, a nano-microfluidic device includes a platform. The platform can include a rigid planar substrate having a surface and with at least one channel, and an aqueous layer in fluid contact with the substrate and the at least one channel. The aqueous layer can contain a fluid and a target.

The substrate can be composed of a material that is IR-reflective, IR-transparent or IR-opaque. In some embodiments, the substrate can be manufactured from silica or silicon, a metal or semiconductor material. In another embodiment, the platform can be a polymer.

The substrate can comprise at least one channel. In some embodiments, the at least one channel can be a well. To create a thin layer of liquid over the target, the target is placed in the at least one channel of the substrate. Fluid flows across the target. In some embodiments, where the target comprises living cells, the fluid can be media providing nutrients and removing waste products from the living cells. In some embodiments, the substrate can have at least 1, at least 5, at least 10, at least 20, at least 25, at least 30, at least 40, at least 50, at least 60, at least 70, at least 80, at least 90, at least 100 channels. While it is generally envisaged that the at least one channel can run parallel to another channel on the substrate, other embodiments are contemplated, for example, a plurality of channels can radiate from a central point on a substrate; and/or bisect one another.

The dimensions of the at least one channel can be determined by a variety of factors. In embodiments where the target comprises living cells, the at least one channel can be large enough to accommodate the cells and allow fluid to flow through the channel, but small enough to reduce the depth of the aqueous layer over the living cells. In some embodiments, cells can include prokaryotic cells which can have diameters such as, for example, 0.5-5 μm. In another embodiments, cells can be those that have formed microbial colonies, biofilms or mat which can have a diameter of, for example, 10-100 μm. In more embodiments, cells can include eukaryotic cells, such as mammalian cells which can have a diameter of, for example, 10-100 μm. As a person with skill in the art will appreciate, the size of a target, for example, a prokaryotic cell, a eukaryotic cell, biofilm, biomat, does not limit any embodiment described herein.

In some embodiments, the at least one channel can have a depth less than 400 nm, less than 500 nm, less than 1 μm, less than 5 μm, less than 10 μm, less than 15 μm, less than 20 μm, less than 25 μm, less than 30 μm, less than 40 μm, less than 50 μm, less than 60 μm, less than 70 μm, less than 80 μm, less than 90 μm, less than 100 μm, and less than 120 μm.

The at least one channel can be formed by any means known in the art, including but not limited to, etching, photolithography, or laser processing. In one embodiment, the at least one channel can be formed by etching, such as Deep Reactive Ion Etch. Any residues can be removed by either wet chemical or dry chemical processes such as resist-strip, oxygen-plasma etch, or RCA cleaning processes.

In some embodiments, the platform includes at least one inlet and at least one outlet. In such embodiments, the at least one inlet and at least one outlet are in fluid communication with the aqueous layer. The fluid of the aqueous layer can flow from the at least one inlet to the at least one outlet. In certain embodiments, the at least one inlet is in fluid communication with an inlet reservoir. In some embodiments, where the target comprises living cells, the inlet reservoir can contain the aqueous medium to sustain and maintain the living cells. The at least one outlet can be in fluid communication with an outlet reservoir. The outlet reservoir can contain fluid that has flowed from the aqueous layer in fluid communication with the substrate. In one embodiment, an additional at least one inlet can be provided to allow the addition of test analytes or reagents to enter the at least one channel. Such test reagents can include any reagent where an effect on the living cells is desired to be observed. Examples of reagents can include small molecules, substrates, stress or anti-stress reagents, O₂ or CO₂, or any analyte, chemical, drug, material or particle for which toxicity measurements are desired. In another embodiment, the media is adjusted to comprise an increased concentration of less volatile constituents that can change the experimental conditions over time. In other embodiments, a test reagent can comprise ionizing radiation.

Substrate coatings

In some embodiments, the substrate can be coated. The coating can cover the inside of the at least one channel and surface of the substrate, or any portions thereof. In further embodiments, the coating can be patterned on the substrate. For example, the inside of the at least one channel can be covered with a coating different from the coating of the remaining surface or portion thereof of the substrate.

The coating can be determined by a variety of factors. For example, the coating can be chosen to reflect electromagnetic radiation from the surface of the substrate, or transmit electromagnetic radiation through the surface of the substrate; modulate the surface tension of the aqueous layer in fluid communication with the substrate; and/or modulate surface profile control. In particular embodiments, the coating can be a thin layer with a depth less than 10 Angstroms, less than 50 Angstroms, less than 100 Angstroms, less than 200 Angstroms, less than 500 Angstroms, less than 1000 Angstroms or less than 5000 Angstroms.

In more embodiments, the coating can be reflective, IR-transparent, IR-semi-transparent, or IR-opaque. In some such embodiments, the coating can be a metal, for example, gold or platinum, or a semiconductor material. IR transparent or semi-transparent materials, can include diamond, ZnSe or Si₃N₄.

The coating can contain a hydrophilic material. Such materials can raise the surface energy sufficiently for the aqueous solution to spread out to a thin film. Example materials include titanium oxide, thiol-terminated chemicals attached to gold surfaces coated surfaces, and gold or platinum films. Titanium oxide can exhibit super-hydrophillic properties after exposure to UV light (Yang, T-S., Shiu, C-B, Wong, M-S, “Structure and hydrophilicity of titanium oxide films prepared by electron beam evaporation”, Surface Science, 548 (2004) 75-82; Maeda, M., Yamasaki, S., “Effect of silica addition of crystallinity and photo-induced hydrophilicity of titania-silica mixed films prepared by sol-gel process”, Thin Solid Films, 483 (2005) 102-106; Premkumar, J., “Highly hydrophilic TiO₂ surface induced by anodic potential”, Chem. Mater. 17 (2005) 944-946). Coatings should be suitable for IR-spectroscopy. In some instances, care can be taken to monitor chemical degradation due to oxidation or surface contamination of the hydrophilic coating.

In another embodiment, the coating can contain a hydrophobic material. Any suitable hydrophobic material known in the art can be used, for example, silicone, SU-8 epoxy, and some polymers, such as Teflon®. In some embodiments, the substrate can be is coated on surfaces outside the channels with the hydrophobic coating to enhance flow of liquid captured in the channel. In one embodiment, during manufacture of the substrate, the substrate can be coated with a photo-patternable material, such as, SU-8 epoxy prior to laser-etching at least one channel in the substrate. In further embodiments, the substrate can be masked to in order to coat specific areas of the substrate, for example, the at least one channel. In such embodiments, the at least one channel can be coated with a reflective or partially reflective coating.

In more embodiments, the surface of the substrate can include structures designed to reduce surface tension of the aqueous layer.

Aqueous layers and targets

In some embodiments, nano-microfluidic devices can include an aqueous layer comprising a fluid and a target. The aqueous layer can be in fluid communication with the substrate. Targets can include any object to be characterized using the nano-microfluidic devices described herein. Typically, targets require a fluid environment to maintain their integrity, for example, an aqueous environment can be required to sustain living cells; and a buffered aqueous environment can be advantageous to maintain the conformation of active proteins such as enzymes. In certain embodiments, targets can include living tissues, living cells, proteins, polymers, and small molecules. The fluid of the aqueous layer can be any fluid required to maintain the integrity of the target. For example, the fluid can comprise water, a simple salt solution, a buffer, a buffer with serum, a medium to maintain cells.

Gas layers

One challenge for systems where the aqueous layer is exposed to the open air, is to prevent contamination by airborne contaminants. In some embodiments, a clean layer of gas can be streamed over the aqueous layer to prevent airborne contaminants entering the aqueous layer. In some embodiments, the gas can be filtered to produce a clean stream of gas. In preferred embodiments, the gas is a sterile gas.

The type of gas utilized can vary with the application of the nano-microfluidic device and can include any source of gas. For example, in some applications where the target is anaerobic bacteria, nitrogen or argon gas can be used to also maintain anaerobic conditions in the aqueous layer. In other example applications, where the target is mammalian cells, the gas can be sterile air enriched with carbon dioxide. In further embodiments, the gas can be a mixture of particular gases.

The rate and content of the gas flow can be adjusted in order to modulate the effects of evaporation on the aqueous layer. In certain embodiments, the gas can be saturated with water to minimize the evaporation along the channel. In some embodiments, the level of water saturation in the gas can be modulated to adjust the levels of evaporation from the aqueous layer, and/or to adjust the levels of humidity above the aqueous layer when measurements are being made. In certain embodiments, evaporation at the outlet can be increased with increased gas flow directed to the outflow in order to increase wicking/drawing of fluid form the aqueous layer. In more embodiments, the gas is dry. The gas flow can be provided and controlled by a variety of means. For example, gas flow can be provided by a fan, such as a microscope fan, and by the controlled release of compressed gas.

In more embodiments, gas can flow above the aqueous layer and below a window.

### Windows

In some embodiments, nano-microfluidic devices can include a window over the aqueous layer. Such windows can have the advantage of reducing the effects of evaporation on the aqueous layer, preventing contamination of the aqueous layer by airborne contaminants, and preventing gaseous exchange between the aqueous layer and the open air.

Windows can be composed of IR-transparent or IR-opaque materials. In one embodiment, the window comprises diamond material which is transparent to a broad spectrum of electromagnetic radiation. Windows composed of such materials can allow measurements to be made from within the aqueous layer while the window is in place above the aqueous layer.

In one embodiment, the window is contacted with a spacer, and the spacer is in contact with the substrate. The spacer is used to determine the distance between the plane of the window and the substrate, as well as the distance between the plane of the window and the aqueous layer. The spacer can have a thickness less than 5 μm, less than 10 μm, less than 15 μm, less than 20 μm, less than 40 μm, less than 100 μm, less than 250 μm, and less than 550 μm. In certain embodiments, the spacer can have an adjustable thickness.

In more embodiments, the window can contact the aqueous layer. In certain embodiments, a gas can flow between the aqueous layer and the window.

In some embodiments, windows may not be desired where the window absorbs or refracts the desired signal, reducing the quality of a signal measurement. Moreover, in some instances, a window can produce undesirable interference patterns due to the inclusion of optical artifacts within the window material. Thus, in some embodiments, the window is removed during measurements.

In other embodiments, a window may be preferred where gaseous exchange between the aqueous layer and the open air is not desired. Examples can include applications where gaseous products dissolved in the aqueous layer are monitored; and where an anaerobic environment in the aqueous layer is desirable.

In more embodiments, where nano-microfluidic devices include windows, the fluid of the aqueous layer can be pumped through the gap between the substrate and the window. In more embodiments, a gas can flow between the window and the aqueous layer. In certain embodiments, the substrate can be raised and lowered with respect to the window in order to modulate the depth of the aqueous layer, and/or the distance between the surface of the aqueous layer and the surface of the window. In such embodiments, the substrate can be coupled to an actuator that raise and lower the substrate with respect to the window.

Heating/cooling systems

In some embodiments, nano-microfluidic devices can include a heating/cooling system. In such embodiments, the heating/cooling system can be used to maintain the temperature of the surface of the substrate and at least one channel. For example, in embodiments where living cells are observed, the temperature of the substrate can be thermally controlled to provide the optimal temperature to maintain cell viability. Such temperatures can be determined by the biological sample, substance, cell or tissue observed on the substrate. For example, temperatures can be maintained within ranges that are between 0-100° C., 1-99° C., 4-70° C., 4-65° C., 10-45° C., 10-42° C., 15-40° C., and 25-40° C. In some embodiments, the temperature can be maintained at 37° C.

Any system known in the art can be used to modulate and maintain the temperature of the substrate. In one embodiment, the temperature control system can include a temperature sensor, processor, and heating/cooling source thermally coupled to the substrate. Heating/cooling sources can include, for example, a heat element, water-filled hoses, a temperature-controlled environment in which the nano-microfluidic device resides, a heat plate below the substrate; and a conductive coating and a heating element connected to the bottom of the platform. In some embodiments, a nano-microfluidic device can be placed in a commercially available microscope environmental chamber.

Sensors and controls

In some embodiments, nano-microfluidic devices can include sensors to monitor conditions on the surface of the substrate. Sensors can detect conditions and changes on the surface of the substrate, and in particular in the at least one channel. Conditions can include, for example, pH, temperature, composition of the aqueous layer, flow of the aqueous layer over the substrate, air-flow over the surface of the substrate, air humidity at the surface of the substrate. Sensors can be embedded in the platform, and in particular, in the substrate.

As will be appreciated by a skilled artisan, sensors can comprise any suitable material, for example, silica, silicon, metal, carbon, or a polymer. In addition, sensors can comprise a variety of shapes and sizes. Examples of components can include, but are not limited to, nanocrystals, nanorods, nanowires, and nanotubes, thin films, and thin layers.

In more embodiments, nano-microfluidic devices can include processors and actuators that can respond to conditions and changes detected by sensors. In one embodiment, a liquid media flow actuator can control fluid flow of the aqueous layer. In some embodiments, such processors and actuators can respond and control the environment of the nano-microfluidic device, in particular, the conditions of the channel, in particular, the conditions of the aqueous layer. Processors can be a component of a computer system. In some embodiments, a computer system can receive data from sensors, process data, record data, and respond to data through actuators and other controls. In one embodiment, a liquid media flow actuator can control fluid flow of the aqueous layer.

In certain embodiments, nano-microfluidic devices can include small-scale sensors. Such small-scale sensors can have dimensions, for example, less than 100 μm, and measure conditions and changes of temperature, pH, composition of the aqueous layer, in particular, the local environment of the biological sample, cells, or living tissues in the at least one channel. Small-scale sensors can be incorporated into the substrate of the platform during manufacture, for example, by using nano-micro-fabrication process to build the sensor within the at least one channel of the substrate, or building the walls of the at least one channel around the small-scale sensor.

IR spectromicroscopy

In some embodiments, any nano-microfluidic device described herein can be utilized in conjunction with systems for performing IR spectroscopy.

Mid-IR spectroscopy offers a rapid, reagentless, and non-destructive analytical technique that can be applied to a wide range of applications in biological systems using the devices and systems disclosed herein. Without wishing to be bound by any one theory, mid-IR spectroscopy can be used to measure the interaction of IR light with particular biomolecules. IR spectroscopy takes advantage of the sensitivity of the mid-IR spectroscopy to the chemical functional groups in molecules. Atoms within a sample-molecule vibrate with characteristic frequencies. A sample-molecule excited by IR light produces an IR signal that can be detected by a detector, such as a mercury-cadmium-telluride detector. Because the sample-molecule absorbs IR light at frequencies where the frequency of the light exactly matches the frequency of the vibration, the IR signal corresponds to the spectrum of IR light absorbed by the sample-molecule. This spectrum can be expressed as a function of the IR light's wavelength (wavenumbers/cm, frequencies/cm). Because the spectrum is unique for every molecular configuration, each mid-IR spectrum of a biological sample represents a “fingerprint” of the chemical functional groups present in a sample-molecule.

The detailed spectrum of a microbial cell was previously thought to be too complex to understand in its totality. However, data in the form of Fourier transform IR (FTIR) spectra can be analyzed using chemometrics. Chemometrics provides a statistical approach to spectral analysis that allows specific spectral features, and their changes, to be correlated with changes in concentration of sample constituents. Example applications of FTIR and chemometrics include: detecting biochemical groups within cellular components, identifying and discriminating bacterial strains of a genus; monitoring population dynamics of microorganisms; characterizing microbial heterogeneity inside a biofilm; quantitating the biodegradable polymer, poly(b-hydroxybutyrate) within bacteria; observing structural changes within bacterial cells; and footprinting metabolites.

Any IR photon source can be used in conjunction with the systems and apparatus described herein. Such IR sources can include, for example, a broad band synchrotron light source, a narrow band light source, a single light source and a multi-wavelength light source. More light sources can include, for example, a synchrotron, a thermal element, a laser, or multiple lasers. In some embodiments the IR photon source irradiates the platform, in particular, the substrate, in particular, the at least one channel, in particular, the target within aqueous layer.

In some embodiments, a detector can detect electromagnetic light, such as IR reflected from the substrate, and/or transmitted through the substrate. In particular embodiments, electromagnetic radiation travels from a source, through a target, such as a cell, and continues to a detector.

In one embodiment, the system includes a FTIR spectrometer, an IR microscope, and any nano-microfluidic device described herein. Because the IR beam does not induce any detectable side-effects in live cells, IR-spectromicroscopy can be used to observe continuously chemical, structural, and conformational changes within target living cells. Example applications further can include observing changes in the chemical, structural and conformational changes in biologically important molecules such as DNA, lipids, proteins and carbohydrates in living cells. Such changes can be observed in many different states of the living cells, for example, during the living cells' response to various stimuli such as small molecules, substrates, and environmental changes. Particular applications can include observing bacterial biologically important molecules under a stress-response event, or observing molecules such as chromates during bioremediation. See methods described in Holman, H.-Y. N., et al., Real-time characterization of biogeochemical reduction of Cr(VI) on basalt surfaces by SR-FTIR imaging. Geomicrobiology Journal, 1999. 16(4): p. 307-324, and co-pending U.S. patent application Ser. No. 10/582,422, entitled “Catheter-Based Mid-IR Reflectance and Reflectance Generated Absorption Spectroscopy,” both of which are hereby incorporated by reference in their entireties.

Methods for maintenance and monitoring of live cells using IR spectroscopy are described in the following references: Holman, H-Y. N., and M. C. Martin. Synchrotron radiation IR spectromicroscopy: a non-invasive molecular probe for biogeochemical processes. Advances in Agronomy, 90: 79-127, 2006; Holman, H. Y. N., et al., Catalysis of PAH biodegradation by humic acid shown in synchrotron IR studies. Environmental Science & Technology, 2002. 36(6): p. 1276-1280; Holman, H.-Y. N., et al., Low-dose responses to 2,3,7,8-tetrachlorodibenzo-p-dioxin in single living human cells measured by synchrotron IR spectromicroscopy. Environmental Science and Technology, 2000. 34(12): p. 2513-2517; and Holman, H.-Y. N., et al., Real-time characterization of biogeochemical reduction of Cr(VI) on basalt surfaces by SR-FTIR imaging. Geomicrobiology Journal, 1999. 16(4): p. 307-324, which are hereby incorporated by reference in their entireties

Fluorescent and visible light microscopy

In some embodiments, the nano-microfluidic system is used for fluorescent microscopy and/or visible light microscopy. With respect to fluorescent microscopy, embodiments can include a fluorescent light source to excite fluorescent labels to be observed, and a detector to capture the emitted light from the excited label. Embodiments of the invention are not limited to any particular analysis technique, but rather include any technique for imaging cells, tissues or biomolecules within the described systems.

Flow cytometers

In one embodiment, a nano-microfluidic device can be coupled to a detector and sorter for use as a flow cytometer. In such embodiments, targets, such as cells, can be suspended in the fluid and flow through the aqueous layer. The aqueous layer can be irradiated with a light source, such as IR light. Reflected, transmitted or absorbed IR-signal from the suspended cells can be detected and used to identify particular characteristics used to sort a plurality of cells suspended in the aqueous layer. A cell sorter downstream of the channel of the nano-microfluidic device can sort the plurality of cells based on information obtained from the reflected, transmitted or absorbed IR-signal.

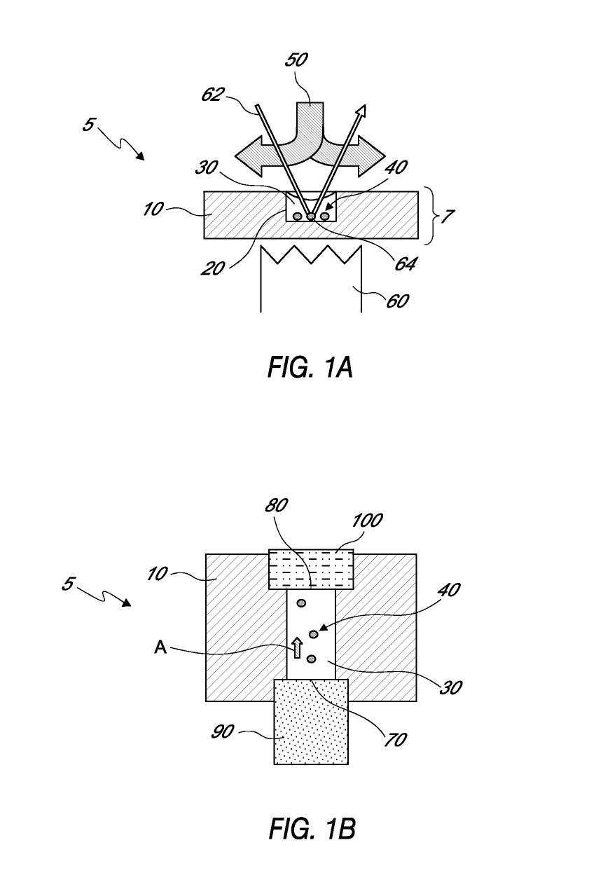

Referring to FIG. 1A, in one embodiment, a nano-microfluidic device (5) can include a platform (7) comprising a planar substrate (10) having a channel (20) and an aqueous layer (30). The channel (20) can have a depth of about 5-10 μm, and width of about 5-100 μm. The aqueous layer (30) contains a liquid that is in fluid contact with the planar substrate (10). As shown, a target (40) is within the channel (20), and inside the aqueous layer (30). In this embodiment, the target (40) is a living cell. A stream of sterile gas (50) flows over the aqueous layer (30), in such a manner to prevent contamination of the aqueous layer by airborne contaminants. As shown, the nano-microfluidic device (5) also includes a source (60) for heating/cooling the device. The source (60) for heating/cooling is in thermal contact with the platform (7).

An infrared light beam (62) is shown reflecting off a target cell (64) as part of any analysis of the target cell (64). As illustrated, the depth of the aqueous layer (30) is sufficient to cover the target cell (64), and there are only tens of nanometers of liquid disposed above the target cell (64) and the top of the aqueous layer (30).

Referring now to FIG. 1B, an inlet (70) and an outlet (80) to the channel (20) are shown in fluid communication with the aqueous layer (30). Fluid in the aqueous layer (30) flows in the direction A from the inlet (70) to the outlet (80). The inlet is in fluid communication with an inlet reservoir (90). The inlet reservoir (90) regulates the flow of fluid into the aqueous layer (30). The outlet (80) is in fluid communication with an outlet reservoir (100), and contains the fluid that is drawn from the aqueous layer (30) through the channel (20).

In one embodiment, the inlet can comprise the end of a capillary tube. In such embodiments, a droplet of fluid can be maintained at the end of the capillary tube by adjusting the pressure of the fluid within the inlet reservoir. For example, a fluid-filled flexible tube can be connected to a capillary tube such that the capillary tube and flexible tube are in fluid communication. By modulating the height of the flexible tube, the pressure of the fluid at the end of the capillary tube can be adjusted to deliver a desired flow of fluid to the aqueous layer within a channel of the device.

In another embodiment, the inlet can comprise the edge of a porous material saturated with fluid, and the inlet reservoir can comprise the porous material saturated with fluid. The porous material can comprise any material suitable for wicking fluid to the aqueous layer. In another embodiment, the outlet can comprise the edge of a porous material, and the outlet reservoir can comprise the porous material unsaturated with fluid.

In yet another embodiment, the inlet can comprise a meniscus at the edge of a solid material placed over the flow channels and media from the meniscus flows into the channels.

In yet another embodiment, the outlet can comprise a non-porous plate suspended over the channel to create a closed capillary channel.

During use, fluid flows from the inlet (70) to the outlet (80) through the aqueous layer in the channel. Fluid can be drawn into the aqueous layer by a variety of means. In some embodiments, capillary action created by the small dimensions of the channel can draw fluid from the inlet and inlet reservoir into the channel and aqueous layer. In more embodiments, fluid can be drawn into the channel (20) to replace fluid that is removed from the aqueous layer.

Fluid can be removed from the aqueous layer by a variety of means. In some embodiments, fluid can be removed by evaporation from the aqueous layer. In more embodiments, the outlet and outlet reservoir can comprise a porous material unsaturated with fluid. In such embodiments, fluid is drawn from the aqueous layer by wicking from the outlet. In more embodiments, the porous material of the outlet reservoir can be warmed. In such embodiments, warming can cause evaporation of fluid and maintain the porous material in a state unsaturated with fluid.

In another embodiment, fluid can be removed by electro-osmotic flow.

In yet another embodiment, fluid can be removed by capillary action.

In various embodiments, the rate of flow of fluid through the aqueous layer can be controlled by modulating factors that can include the rate of evaporation from the aqueous layer; the rate fluid flows through the porous material at the inlet or at the outlet; and dimensions of the channel. In certain embodiment, the apparatus further comprises a pumping source to pump fluid through the aqueous layer. In preferred embodiments, the flow rate can be about 60 μm/sec. However, other flow rates are contemplated.

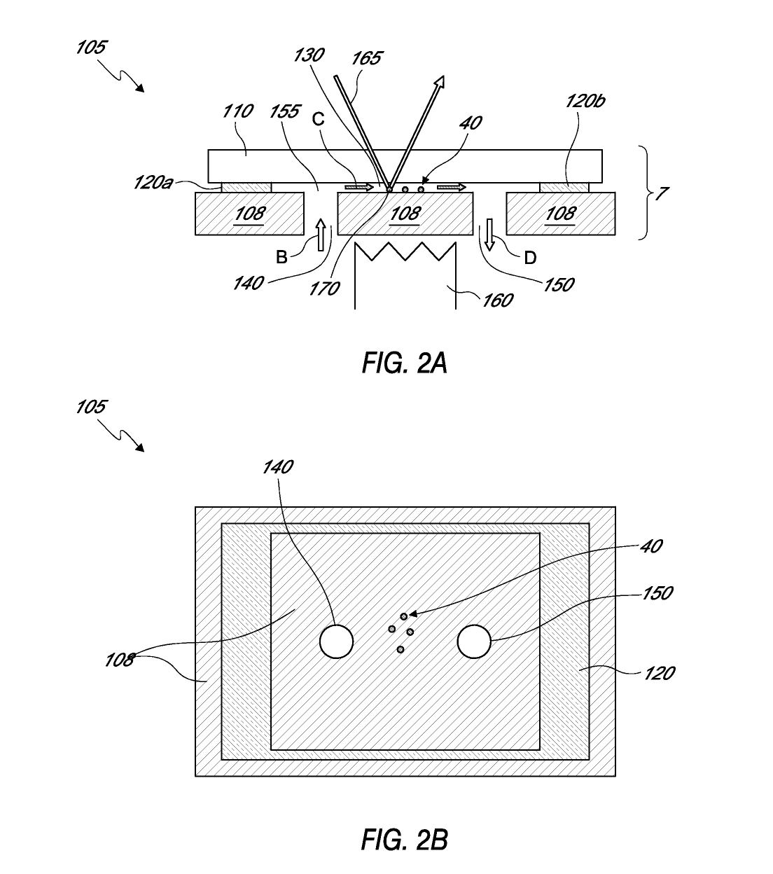

Referring to FIGS. 2A and 2B, in one embodiment, a nano-microfluidic device (105) can include a platform (107) comprising a rigid planar substrate (108), a window (110), and a spacer (120A, 120B). The spacer (120A, 120B) is in contact with the window and the substrate, such that a channel (130) is formed over the substrate. The substrate, an inlet (140), and an outlet (150) are in fluid communication with an aqueous layer (155). The aqueous layer contains fluid and a target (40). In this embodiment, the target is a cell. The spacer can have a thickness of 200 nm to 250 μm. The thickness of the spacer can be adjusted to modulate the depth of the channel created over the substrate. As shown, the nano-microfluidic device includes a source for heating/cooling (160) the device. The source for heating/cooling can be in thermal contact with the platform.

An infrared light beam (165) is shown reflecting off a target cell (170) as part of any analysis of the target cell (170).

During use, fluid can flow in direction B from the inlet through the aqueous layer, and continue to flow through the aqueous layer in direction C, and continue to flow in direction D from the outlet. In some embodiments, fluid can be pumped to or from the aqueous layer via the inlet or outlet, respectively. In more embodiments, the fluid flow can exhibit Hele-Shaw flow.

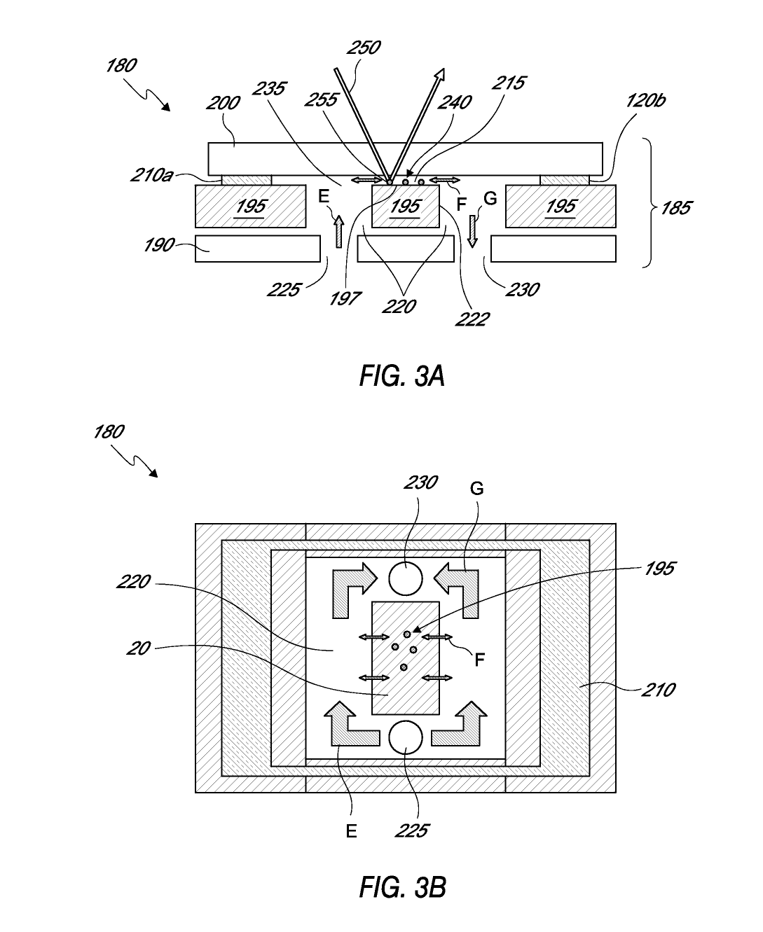

Referring to FIGS. 3A and 3B, a nano-microfluidic device (180) can include a platform (185) comprising a base (190), rigid planar substrate (195) having a surface (197), a window (200), and a spacer (210A, 210B). The spacer is in contact with the window and the base, such that a flow-channel (215) is formed over the surface of the substrate, and a main channel (220) is formed at the circumference of the substrate (222). The surface of the substrate, an inlet (225), and an outlet (230) are in fluid communication with an aqueous layer (235). The aqueous layer contains fluid and a target (240). In this embodiment, the target is a cell. The spacer can have a thickness of 200 nm to 250 μm. An infrared light beam (250) is shown reflecting off a target cell (255) as part of any analysis of the target cell (255).

During use, fluid flows in direction E from the inlet (225) into the main channel (220). Fluid can diffuse in direction F between the main channel (220) and the flow-channel (215), this flow can be Poiseuille flow. Fluid can continue to flow through the aqueous layer to and from the outlet (230) in direction G. In some embodiments where the target is living cells, the flow of fluid through the aqueous layer can supply nutrients to the living cells and remove waste products. The mass flux of the nutrients/waste can be described by:

W=−D(C_{c}−Cₘ)/d

where W is the mass flux of the nutrients/waste, D is the diffusion coefficient or the effective diffusion coefficient for waste substances, Cₑ is the concentration of waste products at the living cells, Cₘ is the concentration of waste products at the media, and d is the depth of the aqueous layer divided by 2.

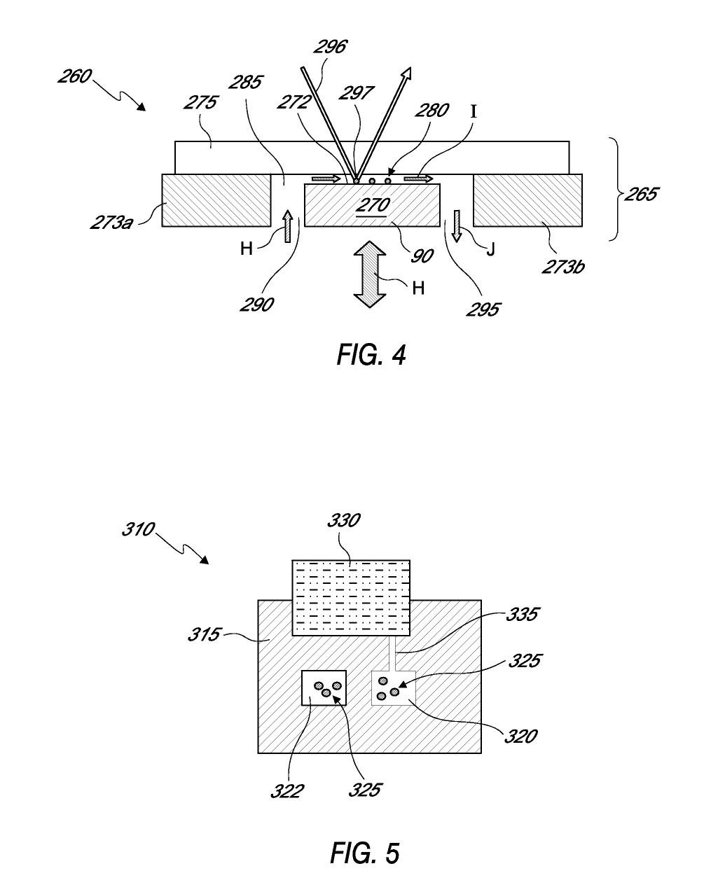

Referring to FIG. 4, in one embodiment, a nano-microfluidic device (260) can comprise a platform (265) including a rigid planar substrate (270) having a surface (272), a spacer (273A, 273B), and a window (275). The spacer (273A, 273B) is in contact with the window (275). A target (280) is in contact with the surface (272) of substrate. In this embodiment, the target (280) is a cell. An aqueous layer (285) comprising a fluid, is in fluid contact with the surface of the substrate, the window, an inlet (290), and outlet (295). An infrared light beam (296) is shown reflecting off a target cell (297) as part of any analysis of the target cell (297).

During use, fluid can flow through the aqueous layer from the inlet (290) in direction H, and continue in direction I, and continue in direction J from the outlet (295). The substrate (270) can be raised or lowered in direction H, thus modulating the depth of the aqueous layer between the window (275) and surface of the substrate (272). One advantage of modulating the depth of the aqueous layer above the surface of the substrate is to reduce IR-absorption by the aqueous layer by raising the substrate while IR-measurements are taken; and to increase flow around a target, such as living cells, by increasing the depth of the aqueous layer when to increase the flow of nutrients to the cells, and flow of waste materials from the cells.

Referring to FIG. 5, in one embodiment, a nano-microfluidic device (310) can include a platform comprising a rigid planar substrate (315) having wells (320, 322). The wells can comprise fluid and a target (325). In this embodiment, the target can be a cell. In this embodiment, a well (320) is in fluid communication with a fluid reservoir (330) through a channel (335). The fluid reservoir can comprise a porous material saturated with fluid.

Although not shown here, the nano-microfluidic device can include a source for heating/cooling (not shown) the device. The source for heating/cooling can be in thermal contact with the platform.

During use, fluid can flow from the fluid reservoir (330) to a well (320) through the channel (335). As fluid evaporates from the well (320), fluid is drawn through the channel from the fluid reservoir.

### EXAMPLES

### Example 1

In one embodiment, a microbial system from each “class” of conditions to be tested is placed on the platform of the apparatus such that temperature, moisture and other experimental conditions can be precisely monitored and controlled by the apparatus. The apparatus is placed on the microscope stage of the IR microscope. The light microscope component of the IR spectral microscope system is used to guide the selection of measurement locations. A single, narrow, or broad band IR beam is directed to the commercial Fourier transform interferometer bench equipped with an IR microscope. After modulation by the interferometer, the modulated IR beam is focused via the IR microscope onto the targeted area inside of the sample using all-reflecting optics. The reflected light from the sample is collected by the microscope optics and sent to the detector(s), which are connected to a computer for collecting spectral data.

The fundamental measurement is a spectrum of reflected, transmitted or absorbed IR. IR spectra can be collected over a wide wavenumber range such as 4000 cm⁻¹ to 650 cm⁻¹. The spectrum for each sampling location at each time point contains at least 8480 data points, each representing an absorbance value at a particular wavelength.

A computer performs a Fourier transform on the measured interferogram to obtain an IR spectrum for each sample location, and removes characteristic CO₂ peaks at 683 cm⁻¹ to 656 cm⁻¹, and 2403 cm⁻¹ to 2272 cm⁻¹, and water vapor fingerprints from the spectra. These data are imported into spectral analysis programs such as Cytospec (version 1) and the Chemometrics Toolbox in MATLAB (version 6) for chemometric analysis. The resulting spectra of reflected, transmitted or absorbed IR are analyzed and the fingerprint spectra are compared to a control. Changes in the spectra are used to detect the presence or effect of various experimental or environmental conditions on live cells, microorganisms, biomolecules and other biological systems.

Live cells can be analyzed to determine enzymatic breakdown or production of cellular byproducts, or the structure or conformation of biological macromolecules and biological water molecule networks. The system can also be used to monitor in real-time cellular organelles such as mitochondria, lysosomes and cellulosomes. In addition, cellular and biological processes can be monitored in real time, in order to evaluate events such as cellular division, differentiation, stress-responses, or adaptive-responses. Thus, methods for observing or monitoring live cells long term are provided using the present apparatus. Furthermore, the IR spectral data obtained from monitoring live cells can be used to provide base line spectral information for normal cells, for comparison to spectral information gathered from cells that are exposed to specific environmental conditions, pollutants, radiation, or test reagents using the present apparatus and methods. Such test reagents can include but are not limited to, stress or anti-stress reagents, toxic gases, suspected carcinogens, or any analyte, chemical, drug, material or particle for which toxicity measurements need to be made.

### Example 2

### Testing Channel Configurations of Nano-microfluidic Devices

Targets were placed in different flow-channel designs, each having a depth of between 5-20 μm, a width of about 40 μm, and length of about 1.5 mm. The flow-channel was designed to be sufficiently deep to completely cover the particular biological particles, yet shallow enough to reduce the absorption of reflected IR signal from the biological particles. The flow-channel was long enough such that wastes accumulated at the flow-channel exit. A sterile flow of nitrogen or argon gas was positioned over the flow-channel to prevent contamination of the sample by airborne contaminants. Fluid-flow through the flow-channel was induced by a combination of slight evaporation, a slight pressure head at the flow-channel entrance, and minor flow-induced wicking at the flow-channel exit which can be enhanced by an additional evaporation at the exit.

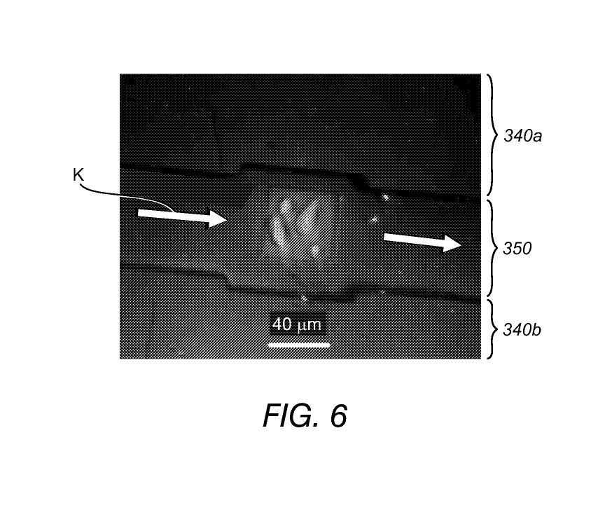

A schematic diagram of the device is shown in FIGS. 1A and 1B, and a picture of the flow-channel is shown in FIG. 6. The flow-channels were fabricated in silicon. Silicon wafers with a diameter of 100 mm were oxidized to a depth of about 150 nm. Flow-channels with a width of 40 μm, depth of 10 μm, and length of 2.5 mm, were etched into the silicon wafer using a deep reactive ion etch process (DRIE). Some flow-channels possessed retention pockets at 250 μm intervals along the length of the flow-channel. These retention pockets measured 50 μm in length and had a width of 50 μm. To enhance IR reflection and measurements of IR reflection, the retention pockets were patterned with thin-film Ti—Au pads.

A deep channel was etched into the silicon wafers at the inlet end and outlet end of the flow-channels. These deep channels had a width of 700 μm wide, depth of 200 μm deep and extended to the wafer's edge. The inlet deep channel was used to seat a glass capillary tube which provided a continuous supply of fluid to the flow-channel. The glass capillary tube had an outer diameter of 656 μm, and an inner diameter of 535 μm. A small drop of silicone was used to hold the capillary tube in the inlet deep channel and to prevent backflow of fluid along the inlet deep channel. The outlet deep channel provided an exit for liquid flowing from the flow-channels. A wicking cloth was placed over the outlet deep channel to absorb fluid flowing from the flow-channels.

The glass capillary tube was about 7 cm in length and was fluidly coupled to a reservoir bottle containing fluid with a 1.5 mm ID Tygon flexible connector tube. The reservoir bottle was placed on a jack-stand, such that the reservoir bottle could be raised or lowered to adjust the head pressure on the exit at the glass capillary tube at the flow-channel.

To initiate flow through the flow-channel, fluid was first siphoned into the connector tubing, and the reservoir bottle was raised until flow was established. To prime the flow into the flow-channels, the flow-channels were temporarily flooded with fluid. The reservoir bottle was lowered to retract the over-spilled fluid on the wafer into the flow channel. As the fluid retracted, fluid was cleared from the wafer surface, but remained in the flow-channel. These steps were repeated to clear any debris lodged in the flow-channel. Following retraction, the reservoir was raised again until a droplet of fluid at the exit of the glass capillary tube was on the verge of advancing. Continuous fluid flow through the flow-channel was established.

The evaporation rate of the fluid flowing through the flow-channel was measured at room temperature. A microcylindrical vessel (diameter 2 mm) was filled with 1 μL of fluid and the time for the volume to evaporate was measured. The measured evaporation rate was 0.005 μL/sec per mm² surface area for water and 0.002 μL/sec per mm² for media. From these evaporation rates, the average rate of flow at the entrance of the flow-channel was approximately 0.1 mm/sec for water and 0.05 mm/sec for media.

FIG. 6 illustrates that E coli reporter strains in the channel show no detectable detrimental effects for incubation in the channel. Microcolonies formed within 24 hours, and cells maintain their membrane integrity for more than 96 hours in the apparatus. Dry sections of the apparatus (340A,340B) are adjacent to the channel (350) containing an aqueous layer and the cells with a fluid flowing with an adjustable flow rate in a direction (K).

### Example 3

### Synchrotron IR Spectromicroscopy to Study of Oxidative Damage

Synchrotron IR spectromicroscopy is used to study oxidative damage in XP-G/CS mutant human fibroblasts. XP-G/CS mutant human cells are devoid of XPG function and unable to carry out TCR. Oxidative damage can arise from attack by reactive oxygen species (ROS), exposure to ionizing radiation or hydrogen peroxide, and metabolism of potential chemical carcinogens. A wide variety of DNA lesions can result from such oxidative damage, for example, base alterations, single-strand breaks, and double-strand breaks (DSBs).

A nano-microfluidic system described herein is used to observe XP-G/CS mutant human cells treated with hydrogen peroxide, or ionizing radiation. Target-cells are placed in channel of nano-microfluidic devices. The devices can maintain the cells at an optimum temperature in an aqueous layer comprising medium supplemented with serum. Several sets of cells are treated with either various doses of hydrogen peroxide, or various doses of ionizing radiation. The cells are observed for several days and measurements are taken using synchrotron IR spectromicroscopy.

Cells treated with hydrogen-peroxide or ionizing radiation have similar DNA mutations, however, cells treated with ionizing radiation also have DSBs. Cells are observed as they recover from treatment. While XP-G/CS cells are more sensitive to hydrogen-peroxide or ionizing radiation, the manner of resulting cell death is not known. Synchrotron IR spectromicroscopy is used to differentiate between the type of cell death, namely, apoptosis vs. necrosis. To characterize the mechanisms of DNA repair in XP-G/CS cells, repair of DNA damage is observed in treated XP-G/CS cells and control cells. Accordingly, nano-microfluidic devices allow continuous observations in real-time of intra-cellular events over an extended period of time.

The above description discloses subject matter including several embodiments for apparatus, systems and methods. This subject matter is susceptible to modification in the methods and materials, as well as alterations in the fabrication methods and equipment. Such modifications will become apparent to those skilled in the art from a consideration of this disclosure or practice of the embodiments disclosed herein. Consequently, it is not intended that this invention be limited to the specific embodiments disclosed herein, but that it cover all modifications and alternatives coming within the true scope and spirit of the invention.

All references cited herein including, but not limited to, published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

What is claimed is:

1. A nano-microfluidic system comprising:
(a) a platform comprising:
(i) a substrate having at least one channel configured to hold at least one cell; and
(ii) an aqueous layer in fluid contact with said substrate, wherein said aqueous layer comprises a fluid that covers said cell by less than 15 μm;
(b) at least one inlet in fluid communication with said aqueous layer; and
(d) at least one outlet in fluid communication with said aqueous layer;
wherein said aqueous layer comprises a fluid that flows from said at least one inlet to said at least one outlet.

2. The system of claim 1, wherein said at least one channel has a depth less than about 100 μm.

3. The system of claim 1, wherein said at least one channel has a depth less than about 10 μm.

4. The system of claim 1, wherein said at least one channel has a depth less than about 5 μm.

5. The system of claim 1, wherein said at least one channel has a depth less than about 1 μm.

6. The system of claim 1, wherein said aqueous layer has a depth less than about 10 μm.

7. The system of claim 1, wherein said aqueous layer has a depth less than about 5 μm.

8. The system of claim 1, wherein said aqueous layer has a depth less than about 1500 nm.

9. The system of claim 1, wherein said aqueous layer has a depth less than about 400 nm.

10. The system of claim 1, wherein said inlet is fluidly coupled to an inlet reservoir.

11. The system of claim 1, wherein said outlet is fluidly coupled to an outlet reservoir.

12. The system of claim 1, wherein said substrate comprises an infrared (IR) transparent material.

13. The system of claim 12, wherein said IR transparent material is selected from the group consisting of diamond, ZnSe, and Si₃N₄.

14. The system of claim 1, further comprising a coating on said substrate.

15. The system of claim 14, wherein said coating is reflective to IR.

16. The system of claim 14, wherein said coating is patterned on said substrate.

17. The system of claim 14, wherein said coating comprises a material selected from the group consisting of titanium oxide, gold, and platinum.

18. The system of claim 14, wherein said coating comprises a material selected from the group consisting of silicone, SU-8 epoxy, and Teflon®.

19. The system of claim 1, further comprising a stream of gas flowing above said aqueous layer.

20. The system of claim 19, wherein said gas is selected from the group consisting of nitrogen, argon, carbon dioxide, air, and mixtures thereof.

21. The system of claim 1, wherein said substrate is in thermal contact with a heating/cooling source.

22. The system of claim 1, further comprising a source of IR irradiating said substrate.

23. The system of claim 22, further comprising a detector of reflected light or transmitted electromagnetic radiation.

24. The system of claim 1, further comprising a window above said substrate.

25. The system of claim 24, further comprising a spacer in contact with said window and said substrate.

26. The system of claim 25, wherein said spacer has a thickness less than 250 μm.

27. The system of claim 25, wherein said spacer has an adjustable thickness.
